(19) United States
(12) Patent Application Publication
(10) Pub. No.: US 2006/0128759 A1
(43) Pub. Date: Jun. 15, 2006

(54) Substituted isoxazole derivatives and their use in pharmaceutics

(75) Inventors: Stefan Laufer, Bleubeuren (DE); Hans-Gunter Striegel, Blaustein (DE); Wolfgang Albrecht, Ulm (DE); Karola Tollmann, Brechen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: MERCKLE GMBH, ULM (DE)

(21) Appl. No.: 10/524,839

(22) PCT Filed: Aug. 19, 2003
(86) PCT No.: PCT/EP03/09191

§ 371 Date: Sep. 13, 2005

(30) Foreign Application Priority Data
Aug. 19, 2002 (DE) 102 37 883.5

(51) Int. Cl. A61K 31/4439; A61K 31/42; C07D 413/02; C07D 261/04

(52) U.S. Cl. 514/340; 514/378; 546/272.1; 548/245

(57) ABSTRACT

The invention relates to substituted isoxazole derivatives of formula (I), wherein the radicals R¹, R² and R³ have the meanings as cited in the description. The inventive compounds comprise an immunomodulatory action and/or an action that inhibits the release of cytokines and are thus suited for treating diseases associated with a disorder of the immune system, particularly immunologically mediated inflammatory diseases

The present invention relates to substituted isoxazole derivatives having immunomodulating and cytokine-release-inhibiting action, to pharmaceutical compositions comprising these compounds and to their use in pharmaceutics.

Pharmacologically active imidazole and isoxazole compounds having anti-inflammatory action are already known. Such imidazole compounds are described, for example, in WO 93/14081. WO 99/03837 describes substituted isoxazoles which inhibit the synthesis of a number of inflammatory cytokines. WO 95/13067 describes oxazole compounds suitable for treating cytokine-mediated diseases. WO 01/12621 describes isoxazole compounds which inhibit c-JUN N-terminal kinases and other protein kinases. Further isoxazole compounds are described in JP 2000-86657, Arzneim.-Forsch./Drug-Res. 43(I), 1993, 441-444, Arch. Pharm. 321, 163-166, 1988, J. Org. Chem. 1985, 50, 2372-2375, Gazz. Chim. Ital., 120, 1990, 1-7 and Chemiker-Zeitung 113, 220-222, 1989.

In spite of the fact that there are known compounds, there is therefore still a need for compounds having anti-inflammatory action which inhibit cytokine release.

It is an object of the invention to provide such compounds.

Surprisingly, it has now been found that certain substituted isoxazole derivatives have high immunomodulating and/or cytokine-release-inhibiting activity.

Accordingly, the present invention provides the substituted isoxazole derivatives of the formula I 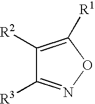
in which
R¹ is selected from the group consisting of
a) H;
b) C₁-C₆-alkyl which may have 1 or 2 substituents independently of one another selected from the group consisting of NR⁴R⁵ and OR⁶;
c) an aromatic or nonaromatic heterocycle having 5 or 6 ring atoms, including 1, 2 or 3 heteroatoms, independently of one another selected from the group consisting of N, O and S, where the heterocycle may have 1 or 2 substituents independently of one another selected from the group consisting of C₁-C₆-alkyl, halogen, CF₃, OR⁶, NR⁷R⁸, —NR⁹COR¹⁰, a radical of the formula II 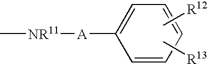
   or a radical of the formula III 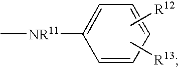
d) phenyl which may have 1, 2 or 3 substituents independently of one another selected from the group consisting of NR⁷R⁸ OR⁶, C₁-C₆-alkyl, halogen, CF₃, CN, NO₂ and CO₂R⁶;
e) phenyl-C₁-C₄-alkyl;
f) C₃-C₈-cycloalkyl; and
g) NR⁷R⁸;
   one of the radicals R² and R³ is a radical of the formula IV 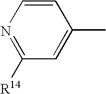
   in which R¹⁴ is C₁-C₆-alkyl, halogen, CF₃, OR⁶, NR⁷R⁸, NR⁹COR¹⁰, a radical of the formula 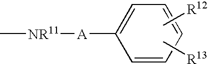
   or a radical of the formula 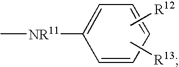
   and
   the second of the radicals R² and R³ is 4-fluorophenyl, 3-trifluoromethylphenyl or 4-trifluoromethylphenyl;
   R⁴ and RS independently of one another are C₁-C₆-alkyl, phenyl or phenyl-C₁-C₄-alkyl or together with the nitrogen atom to which they are attached form a saturated 5- or 6-membered heterocycle having 1 or 2 heteroatoms independently of one another selected from the group consisting of N and O;
   R⁶, R⁷ and R⁸ independently of one another are H or C₁-C₆-alkyl;
   R⁹ is H, C₁-C₆-alkyl or benzyl;
   R¹⁰ is C₁-C₆-alkyl, C₃-C₆-cycloalkyl or phenyl which may have 1 or 2 substituents independently of one another, selected from the group consisting of C₁-C₆-alkyl, C₁-C₆-alkoxy and halogen;
   R¹¹ is H, C₁-C₆-alkyl or phenyl-C₁-C₄-alkyl;
   R¹² and R¹³ independently of one another are H, halogen, C₁-C₆-alkyl or C₁-C₆-alkoxy; and
   A is straight-chain or branched C₁-C₆-alkylene; and
   their optical isomers and physiologically acceptable salts.

The term “alkyl” (also in combination with other groups, such as phenylalkyl, alkoxy, etc.) embraces straight-chain and branched alkyl groups having 1 to 6, preferably 1 to 4, carbon atoms, such as methyl, ethyl, n- and isopropyl, n-, iso- and t-butyl, sec-butyl, n-pentyl and n-hexyl.

The term “aryl” embraces aromatic ring systems, such as phenyl or naphthyl.

The term “halogen” represents a fluorine, chlorine, bromine or iodine atom, in particular a fluorine or chlorine atom.

C₃-C₆-cycloalkyl groups are cyclopropyl, cyclobutyl and, in particular, cyclopentyl and cyclohexyl.

Nonaromatic heterocyclic radicals can be saturated or unsaturated. Preference is given to piperidinyl, piperazinyl, pyranyl, morpholinyl or pyrrolidinyl, where the piperidinyl radical may be substituted by 1, 2, 3 or 4 C₁-C₄-alkyl groups, in particular methyl groups. If R⁴ and R⁵ represent a saturated heterocycle, they are preferably identical radicals.

Preferred aromatic heterocyclic radicals are 2-, 3- or 4-pyridyl, pyrimidinyl, pyrrolyl, imidazolyl, oxazolyl, isoxazolyl, furyl, thienyl or thiazolyl. The heterocyclic radical can be substituted as indicated above.

Phenyl-C₁-C₄-alkyl is in particular benzyl or phenylethyl.

If R¹ represents an aromatic or nonaromatic heterocyclic radical, this is preferably attached via a carbon atom to the isoxazole. It is preferably an aromatic radical, in particular furyl or pyridyl, 4-pyridyl being preferred. The pyridyl radical may be unsubstituted or substituted by NR⁹COR¹⁰, in particular in the 2-position.

If R¹ represents C₁-C₆-alkyl which is substituted by NR⁴R⁵, where R⁴ and R⁵ together with the nitrogen atom to which they are attached, form a saturated heterocycle, this is preferably a radical of the formula V 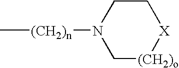
in which X is CH₂, O or N, n is 1 to 6 and o is 0 or 1.

A preferred embodiment are compounds of the formula Ia 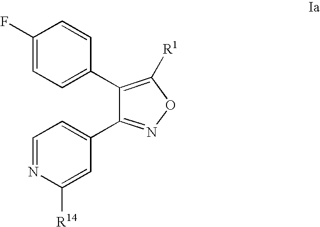
in which R¹⁴ has the meanings given above and represents in particular H, halogen, OR⁶, NR⁷R⁸, NR⁹COR¹⁰, a radical of the formula II 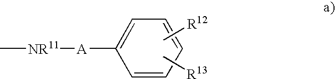
or a radical of the formula III 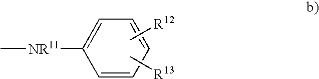
where R⁶ to R¹³ and A have the meanings given above.

In the compounds of the formula Ia, R¹ preferably represents C₁-C₆-alkyl, an aromatic heterocyclic radical having 5 or 6 ring atoms including 1 or 2 heteroatoms independently of one another selected from the group consisting of N and O, where the heterocycle may have 1 or 2 substituents independently of one another selected from the group consisting of halogen, NR⁷R⁸ and NR⁹COR¹⁰, where R⁶ to R¹⁰ have the meanings given above, alkyl which is substituted by NR⁴R⁵ and/or OR⁶, phenyl which is substituted by C₁-C₆-alkoxy and/or NR⁷R⁸, C₃-C₆-cycloalkyl or NR⁴R⁵.

A further preferred embodiment are the compounds of the formula Ib 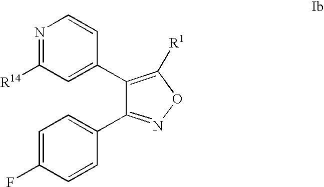
in which R¹⁴ has the meanings mentioned above in connection with formula Ia. In the compounds of the formula Ib, R¹ preferably represents H, C₁-C₆-alkyl, phenyl which is optionally substituted by halogen, in particular in the 4-position, or NR⁴R⁵.

If the compounds according to the invention have centers of asymmetry, the scope of the invention includes both racemates and optical isomers (enantiomers, diastereomers).

In the present case, the physiologically acceptable salts can be acid addition salts or base addition salts. For acid addition salts, inorganic acids, such as hydrochloric acid, sulfuric acid or phosphoric acid, or organic acids, such as tartaric acid, citric acid, maleic acid, fumaric acid, malic acid, mandelic acid, ascorbic acid, gluconic acid and the like are used.

The compounds according to the invention are prepared starting with a compound of the formula 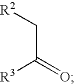
the preparation is illustrated below using the example of R²=4-pyridyl or 4-fluorophenyl and R³=4-fluoro-phenyl or 4-pyridyl, respectively.

The compounds of the formula I in which R² represents an aryl radical are prepared in accordance with scheme 1. The preparation of the compound (3) and its further conversion into the compounds of the formula I is illustrated in more detail in the examples. In this manner, it is possible to prepare the corresponding compounds in which R¹ represents alkyl, substituted alkyl, phenyl, substituted phenyl, phenylalkyl, cycloalkyl and heterocyclyl.

The corresponding regioisomeric compounds can be prepared in accordance with scheme 2. These reactions, too, are illustrated in more detail in the examples.

Compounds of the formula I in which R² represents the pyridyl radical and R¹ represents H or NR⁷R⁸ are prepared in accordance with schemes 3 and 4. The reaction conditions are illustrated in the examples. In this manner, it is also possible to prepare compounds of the formula I in which R¹ represents H or optionally substituted C₁-C₆-alkyl and R¹⁴ represents halogen. Here, the 4-cyanomethylpyridine is replaced by the corresponding 2-halo-4-C¹-C⁶-alkanoylmethylpyridine prepared by reacting 2-halo-4-methylpyridine with lithium diisopropylamide and the corresponding N-methoxymethyl-C₁-C₆-alkanecarboxamide. By substituting the halogen, the resulting compounds can then be converted into other compounds of the formula I, for example into compounds in which R¹⁴ represents OR⁶. The respective reaction conditions are illustrated in the examples.

The preparation of compounds of the formula I in which R² represents an amino- or amido-substituted pyridyl radical is illustrated in scheme 5 using the 4-pyridyl radical as an example. The reactions are described in example 1.

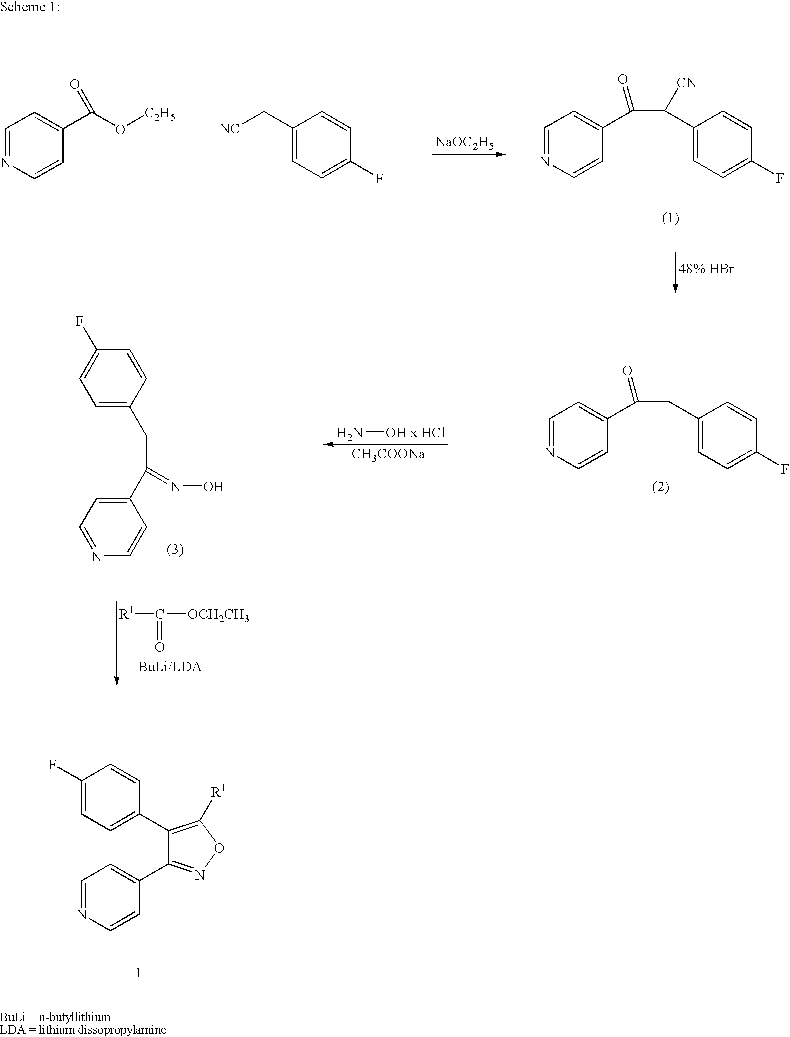

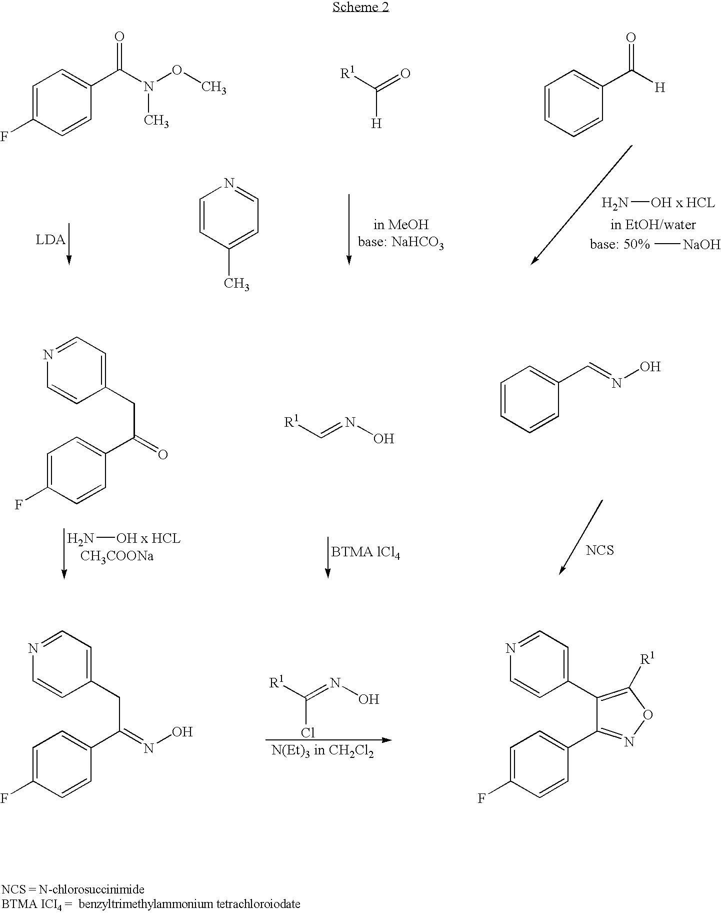

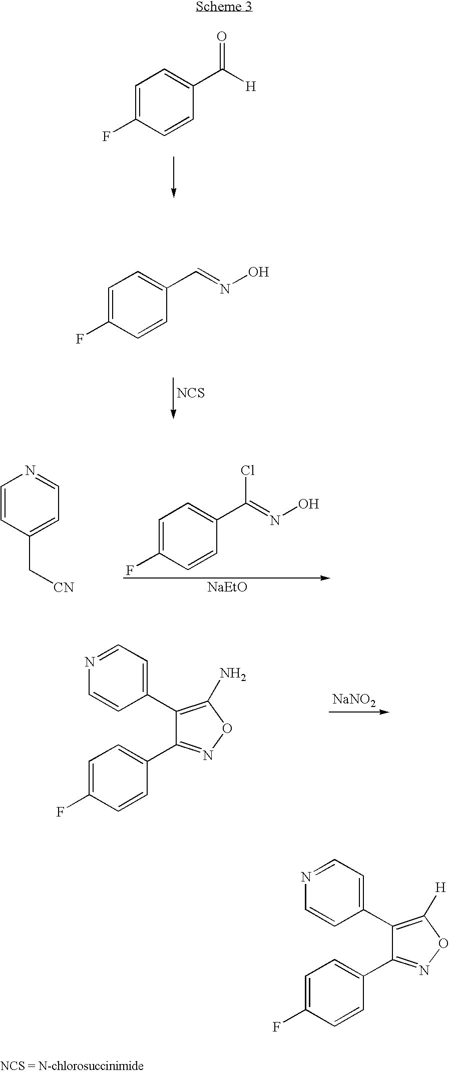

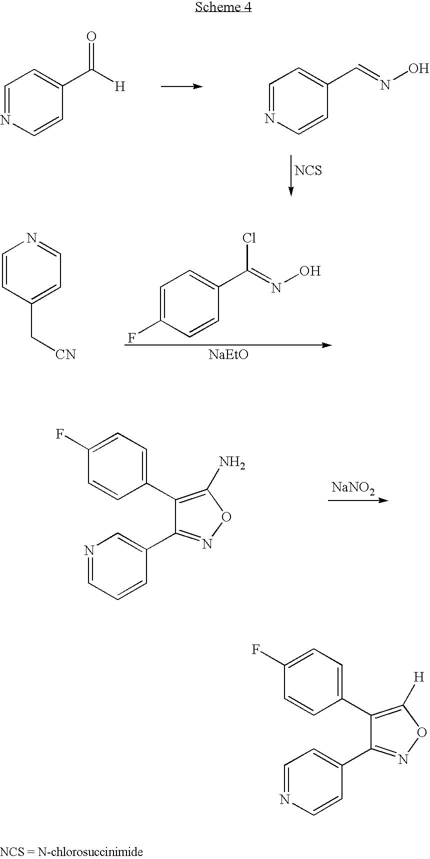

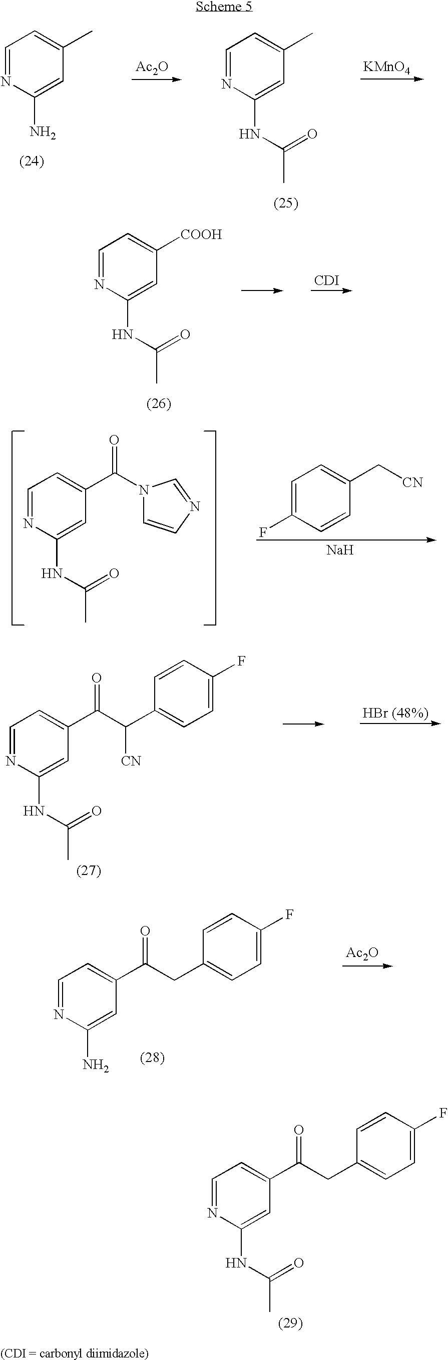

The amino group of the starting material 2-amino-γ-picoline (24) is protected, for example by introducing an acetyl group using acetic anhydride. The methyl group of the compound (25) is subsequently oxidized to the carboxyl group, for example using potassium permanganate in aqueous medium at from 20 to 90° C.

The conversion of the pyridinecarboxylic acid (26) obtained with 4-fluorophenylacetonitrile into compound (27) and the subsequent removal of the nitrile group are carried out in accordance with scheme 1. The acetyl group on the amino group of the pyridine compound is also cleaved off, with formation of the compound (28).

In the next step, the amino group is reprotected, for example by introducing an acetyl group using acetic anhydride. The resulting compound (29) is, according to scheme 1, converted into the compounds of the formula I.

To introduce the desired substituent into the pyridyl group, the acetyl group is initially cleaved hydrolytically using, for example, aqueous acid, which gives the amino compound (35). An acyl radical is introduced by acylation using, in particular, the corresponding acid chloride R¹⁰COCl in an inert solvent, such as an ether, for example tetrahydrofuran, dioxane, or a chlorinated hydrocarbon, for example methylene chloride or 1,2-dichloroethane, etc. The acylation is generally carried out in the presence of a base, for example triethylamine, in an at least equivalent amount.

To prepare the substituted amine compounds, the compound is reacted with one mole equivalent of R—Br, where R is the respective radical to be introduced, in an inert solvent such as dimethylformamide in the presence of a base such as sodium hydride, to give the corresponding monoalkylated or monophenylated compound. If desired, the radical R¹¹ is introduced by reaction with one mole equivalent of R¹¹—Br, under the conditions mentioned.

Alternatively, compounds in which the pyridine radical has an amino substituent can be prepared from the corresponding 5-(halopyridin-4-yl)isoxazole. The process is illustrated in scheme 5 using 2-substituted pyridine compounds where R¹=p-F-phenyl as an example.

The reaction is expediently carried out in the amine in question, which is preferably used in an amount of from 5 to 20 mol equivalents per mole equivalent of the compound (39). The reaction temperature is generally in the range from 100 to 200° C. If desired, it is also possible to employ an inert solvent, such as dioxane, dimethylformamide, etc.

The starting materials (39) can be prepared by the processes described above.

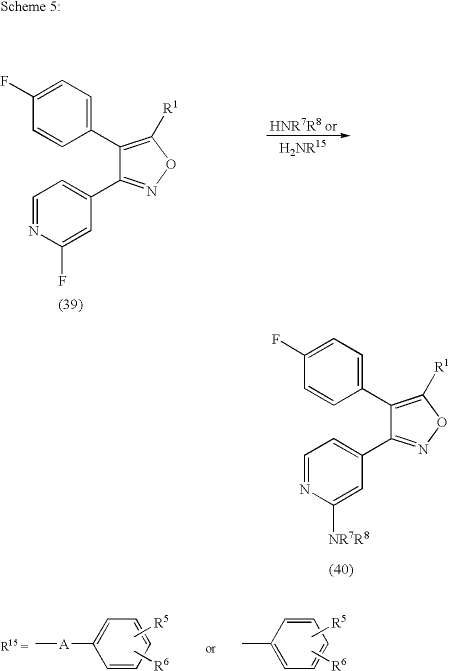

In vitro and in vivo, the compounds according to the invention show immunomodulating and cytokine-release-inhibiting action. Cytokines are proteins such as TNF-α and IL-β which play an important role in numerous inflammatory disorders. The compounds according to the invention are, by virtue of their cytokine-release-inhibiting action, suitable for treating disorders which are associated with a disturbance of the immune system. They are suitable, for example, for treating autoimmune disorders, cancer, rheumatoid arthritis, gout, septic shock, osteoporosis, neuropathic pain, the spread of HIV, HIV dementia, viral myocarditis, insulin-dependent diabetes, periodontal disorders, restenosis, alopecia, T-cell depletion associated with HIV infections or AIDS, psoriasis, acute pancreatitis, rejection reactions of allogenic transplants, allergic pneumonia, arteriosclerosis, multiple sclerosis, cachexia, Alzheimer's disease, stroke, ictus, colitis ulcerosa, Crohn's disease, inflammatory bowel disease (IBD), ischemia, congestive heart failure, pulmonary fibrosis, hepatitis, glioblastoma, Guillain-Barre syndrome, systemic lupus erythematodes, adult respiratory distress syndrome (ARDS) and respiratory distress syndrome.

The compounds according to the invention can be administered either as individual therapeutically active compounds or as mixtures with other therapeutically active compounds. The compounds can be administered on their own; in general, however, they are formulated and administered in the form of pharmaceutical compositions, i.e. as mixtures of the active compounds with suitable pharmaceutical carriers or diluents. The compounds or compositions can be administered orally or parenterally; preferably, they are administered in oral dosage forms.

The type of pharmaceutical composition or carrier or diluent depends on the desired administration form. Oral compositions, for example, can be present as tablets or capsules and may comprise customary excipients, such as binders (for example syrup, gum arabic, gelatin, sorbitol, tragacanth or polyvinylpyrrolidone), fillers (for example lactose, sugar, cornstarch, calcium phosphate, sorbitol or glycine), glidants (for example magnesium stearate, talc, polyethylene glycol or silica), disintegrants (for example starch) or wetting agents (for example sodium lauryl sulfate). Liquid oral preparations can assume the form of aqueous or oily suspensions, solutions, emulsions, syrups, elixirs or sprays and the like. They can also be present as a dry powder which is reconstituted using water or another suitable carrier. Such liquid preparations may comprise customary additives, for example suspending agents, flavors, diluents or emulsifiers. For parenteral administration, it is possible to use solutions or suspensions with customary pharmaceutical carriers.

The compounds or compositions according to the invention can be administered to mammals (man or animal) in a dose of from about 0.5 mg to 100 mg per kg of body weight per day. They may be administered in one individual dose or in a plurality of doses. The activity spectrum of the compounds as inhibitors of cytokine release was examined using the test systems below (C. Donat and S. Laufer in Arch. Pharm. Pharm. Med. Chem. 333, Suppl. 1, 1-40, 2000).

In Vitro Test with Human Whole Blood

The test substance is added to samples of human potassium-EDTA whole blood (of 400 μl each) and the samples are preincubated in a CO₂ incubator (5% CO₂; 95% moisture-saturated air) at 37° C. for 15 min. The samples are then stimulated with 1 μg/ml of LPS (E. coli 026:B6) at 37° C. in a CO₂ incubator (5% CO₂; 95% moisture-saturated air) for 4 hours. The reaction is stopped by placing the samples on ice, adding DPBS buffer and then centrifuging at 1000*g for 15 min. The amount of IL-1β and TNFα in the plasma supernatant is then determined by ELISA.

In Vitro Test with PBMCS

1) The mononuclear cells (PBMCs) from human potassium-EDTA whole blood, diluted 1:3, are isolated by density gradient centrifugation (Histopaque®-1.077). The cells are washed twice with DPBS buffer, resuspended in macrophage SFM medium and adjusted to a cell count of 1*10⁶ cells/ml.
   The resulting PBMCs suspension (samples of in each case 390 μl) and the test substance are preincubated at 37° C. in a CO₂ incubator (5% CO₂; 95% moisture-saturated air) for 15 min. The samples are then stimulated with in each case 1 μl/ml of LPS (E. coli 026:B6) at 37° C. in a CO₂ incubator (5% CO₂; 95% moisture-saturated air) for 4 hours. The reaction is stopped by placing the samples on ice, adding DPBS buffer and then centrifuging at 15 880*g for 12 min. The amount of IL-1β and TNFα in the plasma supernatant is then determined by ELISA.
2) Kinase assay
   At 37° C., microtiter plates were coated for one hour with 50 μl of ATF2 solution (20 μg/ml). The plates were washed three times with water, and 50 μl of kinase mixture (50 mM tris-HCl 10 mM MgCl₂, 10 mM β-glycerol phosphate, 10 μg/ml of BSA, 1 mM DTT, 100 μM ATP, 100 μM Na₃VO₄, 10 ng of activated p38α) with or without inhibitor were added into the wells, and the plates were incubated at 37° C. for 1 hour. The plates were washed three times and then incubated with phosphorus-ATF-2 antibody at 37° C. for one hour. The plates were once more washed three times, and goat-antirabbit IgG labeled with alkaline phosphatase was added at 37° C. for one hour (to fix the antibody-phosphorylated protein/substrate complex). The plates were washed three times, and the alkaline phosphatase/substrate solution (3 mM 4-NPP, 50 mM NaHCO₃, 50 mM MgCl₂, 100 μl/well) was added at 37° C. for 1.5 hours. Formation of 4-nitrophenolate was measured at 405 nm using a microtiter plate reader. The IC₅₀ values were calculated.

The results of the in vitro tests are shown in table 1 below.

TABLE 1
Test results

| | IC₅₀ [mol/1.10⁻⁵] |
|---|---|
| Compound No. | p 38 |
| 1 | 6.75 |
| 2 | — |
| 3 | 3.0 |
| 4 | 0.4 |
| 5 | 2.2 |
| 6 | 2.2 |
| 7 | 2.8 |
| 8 | 2.7 |
| 9 | 1 |
| 10 | 1 |
| 11 | 1 |
| 12 | — |
| 13 | 1.2 |
| 14 | 1) |
| 15 | — |
| 16 | 2.4 · 10⁻² |
| 17 | 2.0 · 10⁻² |

1) 43.6% inhibition at 10⁻⁵ mol/l

### EXAMPLES

### Example 1

### Preparation of the 4-(4-fluorophenyl)-3-(4-pyridinyl)isoxazoles of the formula

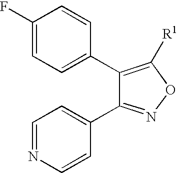

### 2-Cyano-2-(4-fluorophenyl)-1-(4-pyridinyl)ethen-1-ol*HCl (1)

4-Fluorophenylacetonitrile (67.7 g/0.5 mol) and ethyl isonicotinate (75.8 g/0.5 mol) are added dropwise to a 30% strength solution of sodium ethoxide in ethanol (159 g/0.7 mol) and 100 ml of ethanol. The mixture is heated under reflux at boiling point for 30 min, and 1000 g of ice-water are then added. On acidification with HCl_{conc}. to pH 1, the title compound is obtained as a yellow precipitate which is filtered off, washed with H₂O and dried under reduced pressure over P₂O₅.

Yield: 82.94 g/69.1%

Melting point: 225° C.

### 2-(4-Fluorophenyl)-1-(4-pyridinyl)ethanone (2)

The solution of 1 (50 g/0.208 mol) in 48% strength hydrobromic acid (350 ml) is heated under reflux for 20 h. The precipitate 4-fluorophenylacetic acid is filtered off and washed with water. On neutralizing the filtrate with ammonia solution (26%), 2 is obtained as a light-beige precipitate which is filtered off, washed with water and dried over P₂O₅.

Yield: 18.9 g/42.3%

Melting point: 216° C.

¹H-NMR(DMSO): δ(ppm) 4.48 (s, 2H, CH₂), 7.11-7.21 (m, 2H, 4-F-Ph), 7.26-7.34 (m, 2H, 4-F-Ph), 7.89-7.92 (dd, 2H, 4-Pyr), 8.82-8.85 (dd, 2H, Pyr)

### 2-(4-Fluorophenyl)-1-pyridin-4-ylethanone oxime (3)

2 (0.1 mol/21.5 g) is suspended in a 50% strength aqueous methanol solution. After addition of sodium acetate (0.44 mol/36.1 g) and hydroxylamine hydrochloride (0.32 mol/22.0 g), the reaction mixture is heated under reflux for 1.5 h. On cooling in an ice-bath, 3 is obtained as a beige precipitate which is filtered off, washed with water and dried under reduced pressure over P₂O₅.

Yield: 18.1 g/78.5%

Melting point: 154° C.

¹H-NMR(DMSO): δ(ppm) 4.15 (s, 2H, CH₂), 7.04-7.13 (m, 2H, 4-F-Ph), 7.2-7.29 (m, 2H, 4-F-Ph), 7.61-7.64 (dd, 2H, 4-Pyr), 8.53-8.57 (dd, 2H, Pyr), 12.05 (s, 1H, OH)

### General procedure for preparing 4-[4-(4-fluoro-phenyl)isoxazol-3-yl]pyridine of the formula

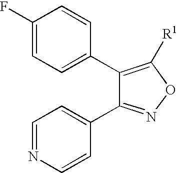

In a three-necked flask flushed with argon, 3 (3.0 g/13 mmol) in 30 ml of THF (tetrahydrofuran) is cooled to −78° C. On dropwise addition of n-butyllithium (15% strength solution in hexane, 24 ml, 55 mmol), there is a temporary temperature increase to −40° C., and the color of the solution turns red. The reaction mixture is stirred at −78° C. for 1 h. The ethyl ester R¹CO₂Et, dissolved in 10 ml of THF, is added dropwise to the reaction mixture: there is a temperature increase to about −55° C. After the addition has ended, the mixture is stirred at −78° C. for 3.5-7 h. On addition of 50 ml of water, the temperature increases and the color of the reaction mixture turns to light green. The mixture is allowed to stand for 30 min and the phases are then separated. The aqueous phase is extracted with 2×50 ml of diethyl ether and allowed to stand overnight. The product crystallizes from the aqueous phase. The organic phases are combined, dried over Na₂SO₄ and concentrated under reduced pressure. If the product does not precipitate from the aqueous phase, it is possible to work up the organic phase by column chromatography (SiO₂ 60, CH₂Cl₂:EtOH=9.5:0.5)

Yields: 2.5-27.7%

The following compounds were obtained by this process:

| Compound | |
|---|---|
| No. | |
| 1 | 4-[4-(4-fluorophenyl)-5-methylisoxazol-3- |
| | yl]pyridine |
| | ¹H-NMR(CDCl₃): δ(ppm) 2.45(s, 3H, CH₃), 7.06-7.20 |
| | (m, 4H, 4-F-Ph), 7.31-7.35(d, 2H, 4- |
| | Pyr), 8.59-8.62(d, 2H, 4-Pyr) |
| 2 | 4-[4-(4-fluorophenyl)-5-pyridinylisoxazol-3- |
| | yl]pyridine |
| | ¹H-NMR(DMSO): δ(ppm)7.16-7.42(m, 4H, 4-Pyr and |
| | 4H, 4-F-Ph), 8.61-8.67(m, 4H, 2×4-Pyr) |
| 3 | N-{4-[4-(4-fluorophenyl)-3-pyridin-4- |
| | ylisoxazol-5-yl]pyridine-2-yl}acetamide |
| | ¹H-NMR(CDCl₃): δ(ppm)2.18 (s, 3H, CH₃), 7.11-7.15 |
| | (m, 1H, 4-Pyr-), 7.23-7.34(m, 4H, 4-F- |
| | Ph and 2H 4-Pyr), 8.0(s, 1H, NH), 8.25-8.28(d, |
| | 1H, 4-Pyr), 8.39(s, 1H, 4-Pyr), 8.59-8.63(dd, |
| | 2H, 4-Pyr) |
| 4 | [4-(4-fluorophenyl)-3-pyridin-4-ylisoxazol-5- |
| | ylmethyl]dimethylamine |
| | ¹H-NMR(DMSO): δ(ppm)2.12/2.15(s, 2×3H, |
| | 2×CH₃), 7.29-7.34(m, 6H, 4-F-Ph and 4-Pyr), |
| | 8.62-8.64(dd, 2H, 4-Pyr) |
| 5 | 4-[4-(4-fluorophenyl)-5-phenylisoxazol-3- |
| | yl]pyridine |
| | ¹H-NMR(CDCl₃): δ(ppm)7.11-7.42(m, 9H, 4-Pyr, |
| | 4-F-Ph and Ar), 7.53-7.58(m, 2H, 4-F-Ph), |
| | 8.60-8.63(dd, 2H, 4-Pyr) |
| 6 | 4-[4-(4-fluorophenyl)-5-furan-2-ylisoxazol-3- |
| | yl]pyridine |
| | ¹H-NMR(DMSO): δ(ppm)6.68-6.71(m, 1H, Fur), |
| | 6.77-6.80(dd, 1H, Fur), 7.30-7.39(m, 4H, 4- |
| | Pyr and 4-F-Ph), 7.44-7.51(m, 2H, 4 F-Ph), |
| | 7.92-7.93(dd, 1H, Fur), 8.63-8.66(dd, 2H, |
| | 4-Pyr) |
| 7 | 4-[5-cyclopropyl-4-(4-fluorophenyl)isozazol- |
| | 3-yl]pyridine |
| | ¹H-NMR(DMSO): δ(ppm)1.02-1.11(m, 4H, |
| | cycloprop), 1.19-2.11(m, ¹H, cycloprop), |
| | 7.24-7.40(m, 6H, 4-F-Ph and 4-Pyr), 8.60-8.63(d, |
| | 2H, 4-Pyr) |
| 8 | {4-[4-(4-fluorophenyl)-3-pyridin-4- |
| | ylisoxazol-5-yl]phenyl}dimethylamine |
| | ¹H-NMR(CDCl₃): δ(ppm)3.03(s, 6H, 2×CH₃), 6.7-6.79(d, |
| | 2H, Ar), 7.18-7.34(m, 4H, 4-F-Ph), |
| | 7.38-7.43(d, 2H, Ar), 7.89-7.91(d, 2H, |
| | Pyr), 8.67-8.70(d, 2H, Pyr) |
| 9 | 4-[4-(4-fluorophenyl)-5-piperidin-1- |
| | ylisoxazol-3-yl]pyridine |
| | ¹H-NMR(CDCl₃): δ(ppm)1.41-1.43(m, 2H, —CH₂), |
| | 1.55-1.63(m, 4H, 2×CH₂—), 3.5(s, 1H, —CH₂—), |
| | 7.06-7.29(m, 4H, 4-F-Ph), 7.32-7.36(dd, 2H, |
| | Pyr), 8.59-8.61(dd, 2H, Pyr) |
| 10 | 4-[4-(4-fluorophenyl)-5-(4-methoxy- |
| | phenyl)isoxazol-3-yl]pyridine |
| | ¹H-NMR(CDCl₃): δ(ppm)3.82(s, 3H, —CH₃), 6.84- |
| | 6.89(m, 2H, Ar), 7.09-7.34(m, 6H, 4-F-Ph |
| | and 4-Pyr), 7.45-7.49(m, 2H, Ar), 8.58-8.61 |
| | (dd, 2H, Pyr) |
| 11 | 4-[5-(ethoxyphenyl)-4-(4-fluorophenyl)- |
| | isoxazol-3-yl]pyridine |
| | ¹H-NMR(CDCl₃): δ(ppm)1.38-1.61(t, 3H, —CH₃), |
| | 3.98-4.09(q, 2H, —CH₂—)6.82-6.87(m, 2H, |
| | Ar), 7.09-7.25(m, 6H, 4-F-Ph and 4-Pyr), |
| | 7.30-7.33(m, 2H, Ar), 8.57-8.6(dd, 2H, Pyr) |
| 12 | 4-[4-(4-fluorophenyl)-5-methoxymethyl- |
| | isoxazol-3-yl]pyridine |
| | ¹H-NMR(CDCl₃): δ(ppm)3.42(s, 3H, —CH₃), 4.48(s, |
| | 2H, —CH₂—)7.06-7.23(m, 4H, 4-F-Ph), |
| | 7.26-7.36(dd, 2H, 4-Pyr), 8.59-8.62(dd, 2H, Pyr) |

### Example 2

### Preparation of 3-(4-fluorophenyl)-4-(4-pyridinyl)isoxazoles of the formula

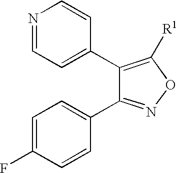

### 4-Fluoro-N-methoxy-N-methylbenzamide (1)

In an ice-bath, a mixture of O,N-dimethylhydroxylamine hydrochloride (9.7 g/0.1 mol) and triethylamine (30.4 ml/0.218 mol) in 165 ml of dichloromethane is cooled to 0° C. and stirred for 1 h. With ice-cooling, 4-fluorobenzoyl chloride (12 ml/0.1 mol) is added dropwise over a period of 15 min. After 2 h, ice-cooling is removed and the mixture is stirred at room temperature for another 1 h. A white suspension is formed, and 100 ml of H₂O are added. The organic phase is separated off and the aqueous phase is extracted with 3×50 ml of diethyl ether. The combined organic extract is dried over Na₂SO₄ and concentrated under reduced pressure. After cooling and scratching, compound 1 crystallizes out.

Yield: 12.5 g/63.23%

¹H-NMR(CDCl₃): δ(ppm) 3.35 (s, 3H, CH₃), 3.53 (s, 3H, OCH₃), 7.04-7.13 (m, 2H, 4-F-Ph), 7.71-7.78 (m, 2H, 4-F-Ph)

### 1-(4-Fluorophenyl)-2-pyridin-4-ylethanone (2)

In a three-necked flask flushed with argon, diisopropylamine (20.5 g/0.2 mmol) is initially charged in 200 ml of THF and the mixture is cooled to −78° C. and stirred for a short while. On dropwise addition of n-butyllithium (15% strength solution in hexane, 91 ml, 0.21 mmol), there is a temporary temperature increase to −40° C. The reaction mixture is stirred at −78° C. for 1 h. A clear light-yellow solution is formed. Picoline (9 g/97 mmol) in 10 ml of THF is added dropwise to the reaction mixture: temperature increase to −55° C. and immediate change of color to red. After the addition has ended, the mixture is stirred at −78° C. for 1 h, and 1 (15 g/82 mmol), dissolved in THF, is added dropwise over a period of 2 min. After a brief temperature increase to −60° C., the reaction mixture is stirred at −78° C. for 1.5 h and then at 0° C. for 1 h. The mixture is poured into a mixture of 100 ml of saturated NaCl solution covered with 100 ml of ethyl acetate. The organic phase is separated off and the aqueous phase is extracted with 3×70 ml of diethyl ether. The combined organic phases are dried over Na₂SO₄ and concentrated under reduced pressure. The orange oily reaction mixture is either purified by column chromatography (EtOH:CH₂Cl₂=0.5:9.5) or reacted further as crude product.

Yield: 8.1 g/38.9%

¹H-NMR(CDCl₃): δ(ppm) 4.27 (s, 2H, CH₂), 7.12-7.21 (m, 4H, 4-F-Ph and 4-Pyr), 7.99-8.07 (m, 2H, 4-F-Ph), 8.56-8.59 (m, 2H, 4-F-Pyr)

### 1-(4-Fluorophenyl)-2-pyridin-4-ylethanone oxime (3)

The compound is prepared analogously to compound 3, example 1.

Yield: 20.7 g/90%

¹H-NMR(CDCl₃): δ(ppm) 4.21 (s, 2H, CH₂), 6.99-7.08 (m, 2H, 4-F-Ph), 7.21-7.27 (dd, 2H, 4-Pyr), 7.54-7.63 (m, 2H, 4-Pyr), 8.49-8.53 (dd, 2H, 4-Pyr), 9.85 (s, 1H, —OH)

### 4-Fluorobenzaldehyde oxime (4A) and benzaldehyde oxime (4B)

150 ml of a 50% strength NaOH solution are added dropwise to a mixture of 60 ml of H₂O+90 ml of ice +60 ml of EtOH, 4-fluorobenzaldehyde (24.5 g/0.2 mol) or benzaldehyde (21.2 g/0.2 mol) and hydroxylamine hydrochloride (19 g/0.27 mol). The reaction mixture is placed into an ice-bath to keep the temperature at <30° C. The mixture is stirred at room temperature for 1 h, cooled in an ice-bath, neutralized to pH 6 using HCl_{conc} and extracted with 2×200 ml of diethyl ether, and the extracts are dried over Na₂SO₄ and concentrated under reduced pressure.

Yield: 4A: 12.6 g/45%
4B: 18.6 g/76.9%

### 4-Fluorobenzylchloromethane oxime (5A) and benzyl-chloromethane oxime (5B)

At room temperature, N-chlorosuccinimide (12 g/0.09 mol) is added as a solid with stirring to a solution of 4A (12.5 g/0.09 mol) or 4B (10.9 g/0.09 mol) in 100 ml of DMF. After addition of 10% of the amount of N-chlorosuccinimide, the gas phase of an HCl_{conc} bottle is bubbled into the reaction mixture to initiate the reaction. On further addition of N-chloro-succinimide, there is a temporary temperature increase to 50° C., and the color of the reaction solution changes to light yellow. After stirring at room temperature (1 h), 300 ml of ice-water are added to the mixture, which is then extracted with 3×100 ml of diethyl ether. The combined diethyl ether phases are dried over Na₂SO₄ and concentrated under reduced pressure.

Yield: 5A: 7.49 g/51%
5B: 13.4 g/95%

### 2-Methanepropane oxime (6)

A mixture of hydroxylamine hydrochloride (7.0 g/0.1 mol) and NaHCO₃ (8.4 g/0.1 mol) is slowly added to a solution of isobutyraldehyde (4.5 ml/0.05 mol) in methanol (150 ml). The reaction mixture is heated under reflux for 45 min and stirred at room temperature for 30 min. The precipitate (NaCl) is filtered off and the filtrate is concentrated under reduced pressure. The colorless oily crude product is used without further work-up.

Yield: 1.36 g/32.2%

### 1-Chloro-2-methylpropane oxime (7)

At 0° C., BTMA ICl₄⁽¹⁾ (12.5 g/0.031 mol) is added as a solid to a solution of 6 (2.7 g/0.031 mol) in 100 ml of CH₂Cl₂. The color of the yellow suspension changes from yellow to orange and then to light green. After 1 h of stirring at 0° C., BTMA ICl₂ is precipitated using 100 ml of diethyl ether. The precipitate is filtered off and the filtrate is concentrated under reduced pressure at 10° C. The oily crude product is used without further work-up.

Yield: 2.1 g/55.7%

### ⁽¹⁾BTMA ICl₄ benzyltrimethylammonium tetrachloroiodate

### 4-[3-(4-Fluorophenyl)-5-phenylisoxazol-4-yl]pyridine (13) and 4-[3,5-bis(4-fluorophenyl)isoxazol-4-yl]pyridine (14)

A solution of 3 (2.3 g/10 mmol) in 100 ml of CH₂Cl₂ is cooled to 0° C., and triethylamine (2.8 g/27 mmol) is added. After 45 min of stirring at 0° C., 5A or 5B (4 g/23 mmol) in 20 ml of CH₂Cl₂ is added dropwise. After 12 stirring at from 0° C. to room temperature, the precipitate (triethylamine×HCl) is filtered off, and the CH₂Cl₂ phase is concentrated using a rotary evaporator. The organic phase is worked-up by column chromatography (SiO₂ 60, CH₂Cl₂:EtOH=9.5:0.5)

Yield: 13A: 0.53 g/17%
13B: 0.43 g/12.9%

### 4-[3-(4-Fluorophenyl)-5-phenylisoxazol-4-yl]pyridine (13)

¹H-NMR(CDCl₃): δ(ppm) 7.06-7.20 (m, 4H, 4-F-Ph and 4-Pyr), 7.38-7.42 (m, 4H, Ph), 7.49-7.54 (m, 2H, 4-F-Ph), 8.63-8.67 (dd, 2H, 4-Pyr)

### 4-[3,5-Bis(4-fluorophenyl)isoxazol-4-yl]pyridine (14)

¹H-NMR(CDCl₃) δ(ppm) 6.99-7.16 (m, 6H, 2×4-F—Ph), 7.32-7.39 (m, 2H, 4-Pyr), 7.46-7.53 (m, 2H, 4-F-Ph), 8.62-8.65 (dd, 2H, 4-Pyr)

### 4-[3-(4-Fluorophenyl)-5-isopropylisoxazol-4-yl]pyridine (15)

A solution of 3 (0.7 g/3.4 mmol) in 50 ml of CH₂Cl₂ is cooled to 0° C., and triethylamine (1.2 g/12 mmol) is added. After 45 min of stirring at 0° C., undiluted 7 (1.8 g/14.9 mmol) is added dropwise. After 12 h of stirring at from 0° C. to room temperature, the precipitate (triethylamine×HCl) is filtered off, and the CH₂Cl₂ phase is concentrated using a rotary evaporator. The organic phase is worked up by column chromatography (SiO₂ 60, CH₂Cl₂:EtOAc=4:6).

Yield: 0.043 g

¹H-NMR(CDCl₃): δ(ppm) 1.34 (s, 3H, —CH₃), 1.37 (s, 3H, —CH₃), 3.13-3.20 (m, 1H, CH) 6.97-7.1 (m, 4H, 4-F-Ph and 4-Pyr), 7.32-7.39 (m, 2H, 4-Pyr), 8.59-8.63 (dd, 2H, 4-Pyr)

### Example 3

### Preparation of 3-(4-fluorophenyl)-4-(4-pyridinyl)-isoxazoles of the formula

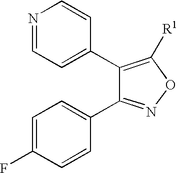

### 3-(4-Fluorophenyl)-4-pyridin-4-ylisoxazol-5-ylamine (16)

At room temperature, a solution of NaEtOH (1.7 g/0.025 mol) in 40 ml of EtOH is added to a solution of 4-pyridinylacetonitrile (2.97 g/0.025 mol) in THF. The reaction mixture is cooled to 0° C., and 4-fluorobenzylchloromethane oxime, dissolved in ethanol, is then added dropwise over a period of 10 min, and stirring at 0° C. is continued for 1 h. The mixture is then heated at 45° C. for 1 h and concentrated using a rotary evaporator and then taken up in 200 ml of water, and CH₂Cl₂ is added. The product 16 is obtained as a red precipitate.

Yield: 3.05 g/47.8%

¹H-NMR(CDCl₃): δ(ppm) 4.91 (s, 2H, NH₂), exchangeable), 7.03-7.12 (m, 4H, 4-F-Ph and 4-Pyr), 7.38-7.45 (m, 2H, 4-F-Ph), 8.53-8.56 (dd, 2H, 4-Pyr)

### 4-[3-(4-Fluorophenyl)isoxazol-4-yl]pyridine (17)

16 (0.7 g/2.74 mmol) is dissolved in a mixture of 20 ml of glacial acetic acid, 10 ml of H₂O and 10 ml of THF. At room temperature, NaNO₂ (1.9 g/27.4 mmol) is added a little at a time over a period of 1 h. After 30 min of stirring, the mixture is diluted with water and extracted with 3×50 ml of CH₂Cl₂. The combined organic phases are dried over Na₂SO₄ and concentrated under reduced pressure. The product is purified by column chromatography (EtOAc:CH₂Cl₂=7:3). The main product formed is 4-(4-fluorophenylethynyl)pyridine.

Yield: 51.54 mg/7.84%

¹H-NMR(CDCl₃): δ(ppm) 7.08-7.20 (m, 4H, 4-F-Ph and 4-Pyr), 7.44-7.51 (m, 2H, 4-F-Ph), 8.60-8.62 (dd, 2H, 4-Pyr), 8.67 (s, 1H, —CH)

⁽¹⁾BTMA×ICl₄: benzyltrimethylammonium tetrachloroiodate

### Example 4

### 4-(4-Fluorophenyl)-3-(4-pyridinyl)isoxazole

### Chloropyridinylmethane oxime (1)

At 0° C., BTMA ICl₄⁽¹⁾ (8.38 g/0.02 mol) is added as a solid to a solution of 4-pyridinaldoxime (2.5 g/0.02 mol) in 100 ml of CH₂Cl₂. Simultaneously to a slight temperature increase, the color of the yellow suspension changes to orange. After 6 hours of stirring at room temperature, the precipitate of 1 is filtered off.

Yield: 2.9 g/95%

¹H-NMR(DMSO) δ(ppm) 8.12-8.15 (dd, 2H, 4-Pyr), 8.87-8.90 (dd, 2H, 4-Pyr), 13.6 (s, 1H, OH)

### 4-(4-Fluorophenyl)-3-pyridin-4-ylisozazol-5-ylamine (18)

At room temperature, a solution of NaOEt (0.34 g/5 mol) in 10 ml of EtOH is added to a solution of 4-fluoro-phenylacetonitrile (0.68 g/5 mmol) in DMF (dimethyl-formamide), the mixture is stirred for 30 min, 1 (0.785 g/5 mmol), dissolved in DMF, is added dropwise over a period of 10 min and stirring at room temperature is continued for another 6 h. The mixture is taken up in 100 ml of water and extracted with 3×50 ml of CH₂Cl₂, and the extracts are dried over Na₂SO₄ and concentrated under reduced pressure. The reaction mixture is worked up by column chromatography (EtOAc:CH₂Cl₂=6:4).

Yield: 20 mg

¹H-NMR(CDCl₃): δ(ppm) 4.66 (s, 2H, NH2), 7.06-7.22 (m, 4H, 4-F-Ph), 7.34-7.37 (dd, 2H, 4-Pyr), 8.59-8.62 (dd, 2H, 4-Pyr)

### 4-[4-(4-Fluorophenyl)isoxazol-3-yl]pyridine (19)

The compound is prepared analogously to (17), example 3.

Yield: 110 mg/29%

¹H-NMR(CDCl₃): δ(ppm): 7.05-7.14 (m, 2H, 4-F-Ph); 7.20-7.24 (m, 2H, 4-F-Ph); 7.39-7.42 (dd, 2H, 4-Pyr); 8.56 (s, 1H, C5); 8.64-8.67 (dd, 2H, 4-Pyr)

### Example 5

### 4-Fluorobenzaldehyde oxime (1)

Hydroxylamine hydrochloride (19 g/270 mmol) is added to a mixture of 4-fluorobenzaldehyde (24.2 g, 200 mmol) in 60 ml of water, 90 ml of ice and 60 ml of ethanol. With stirring, 150 ml of a 50% strength NaOH solution are added dropwise. The reaction mixture is placed into an ice-bath to keep the temperature during the dropwise addition at <30° C. The mixture is then stirred at room temperature for another 1 h. Neutralization with concentrated hydrochloric acid results in the formation of a white precipitate, which is extracted using 2×200 ml of diethyl ether. The organic phases are dried over Na₂SO₄ and concentrated under reduced pressure. The title compound is obtained as a white precipitate.

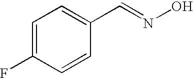

Yield: 12.6 g/45%

¹H-NMR(CDCl₃): δ(ppm) 7.05-7.15 (m, 2H, 4-F-Phe), 7.54-7.61 (m, 2H, 4-F-Phe), 8.14 (s, 1H, —CH), hydroxyl group not visible

### 4-Fluorobenzylchloromethane oxime (2)

At room temperature, N-chlorosuccinimide (12 g, 90 mmol) is, as a solid, added with stirring to a solution of 1 (12.5 g, 90 mmol) in 100 ml of DMF. About 10% of the amount of N-chlorosuccinimide are added, and gaseous HCl is then bubbled through the mixture to initiate the reaction. During the addition of more N-chlorosuccinimide, there is a temporary temperature increase to 50° C., and the color of the reaction solution changes to light yellow. After 1 h of stirring at room temperature, 300 ml of ice-water are added to the mixture, and the mixture is extracted with 3×100 ml of diethyl ether. The combined diethyl ether phases are dried over Na₂SO₄ and concentrated under reduced pressure. The title compound crystallizes in a freezer.

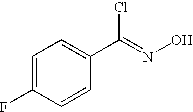

Yield: 7.49 g/51%

¹H-NMR(CDCl₃): δ(ppm) 7.01-7.1 (m, 2H, 4-F-Phe), 7.77-7.85 (m, 2H, 4-F-Phe), 10.3-10.8 (s, 1H, —OH, exchangeable)

### 1-(2-Fluoropyridin-4-yl)propan-2-one (3a)

In a three-necked flask flushed with argon, diiso-propylamine (2.9 ml, 20 mmol) is initially charged in 30 ml of THF_{dist}, and the mixture is cooled to −78° C. and stirred for a short while. On dropwise addition of n-butyllithium (15% strength solution in hexane, 9.1 ml, 21 mmol), there is a temporary temperature increase to −40° C. The reaction mixture is stirred at −78° C. for 30 h. A clear light-yellow solution is formed. 2-Fluoro-4-methylpyridine (2.2 g, 20 mmol) in 3 ml of THF_{dist} is added dropwise to the mixture: temperature increase to −55° C. After the addition has ended, the mixture is stirred at −78° C. for 45 min, and N-methoxymethylacetamide (2.06 g, 20 mmol) is added dropwise. After a brief temperature increase to −60° C., the reaction mixture is stirred at −78° C. for 3 h. The mixture is taken up in 50 ml of water and stirred for 30 min, until it has reached room temperature. The organic phase is separated off, and the aqueous phase is extracted with 2×50 ml of diethyl ether. The combined organic phases are dried over Na₂SO₄ and concentrated under reduced pressure.

The orange, oily reaction mixture is purified by column chromatography.

Yield: 250 mg/8.1%

MS m/z (%) 153, 171, 156, 91, 77, 64, 61

¹H-NMR(CDCl₃): δ(ppm) 2.17 (s, 3H, —CH₃), 3.73 (s, 2H, —CH₂—), 6.72 (s, 1H, 4-Pyr), 6.93-6.98 (dd, 1H, 4-Pyr), 8.07-8.1 (dd, 1H, 4-Pyr)

### 1-(2-Bromopyridin-4-yl)propan-2-one (3b)

3b is prepared from 2-bromo-4-methylpyridine (3.44 g, 20 mmol) using the synthesis described for 3a.

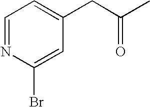

Yield: 350 mg/8.1%

MS m/z (%) 214, 171, 156, 91, 77, 64, 61

¹H-NMR(CDCl₃): δ(ppm) 2.22 (s, 3H, —CH₃), 3.69 (s, 2H, —CH₂—), 7.06-7.09 (dd, 1H, 4-Pyr), 7.33 (s, 1H, 4-Pyr), 8.27-8.3 (dd, 1H, 4-Pyr)

### 2-Fluoro-4-[3-(4-fluorophenyl)-5-methylisoxazol-4-yl]pyridine (20)

1-(2-Fluoropyridin-4-yl)propan-2-one 3a (0.25 g, 1.6 mmol) is dissolved in ethanol, and 10 drops of triethylamine are then added dropwise and the mixture is stirred for a short while at room temperature. 4-Fluorobenzylchloromethane oxime 2 (0.4 g, 2.8 mmol) is added, and the mixture is then heated under reflux for 16 h. The mixture is concentrated using a rotary evaporator, taken up in water and extracted with 3×50 ml of dichloromethane. The combined organic phases are dried over Na₂SO₄ and concentrated under reduced pressure.

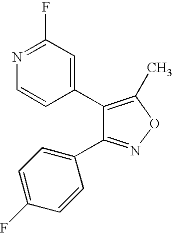

MS m/z (%) 272, 257, 240, 209, 108, 123, 95, 83

### 2-Bromo-4-[3-(4-fluorophenyl)-5-methylisoxazol-4-yl]pyridine (21)

3b is prepared from 1-(2-bromopyridin-4-yl)propan-2-one 3b (0.35 g, 1.6 mmol) according to the synthesis described for 20.

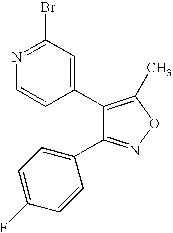

MS m/z (%) 334, 317, 290, 210, 184, 170, 184, 95, 75

¹H-NMR(CDCl₃): δ(ppm) 2.51 (s, 3H, —CH₃), 6.98-7.11 (m, 1H, 4-Pyr and 2H, 4-F-Phe), 7.31-7.41 (m, 1H, 4-Pyr and 2H, 4-F-Phe), 8.28-8.36 (dd, 1H, 4-Pyr)

### 2-Chloro-4-[3-(4-fluorophenyl)-5-methylisoxazol-4-yl]pyridine (22)

20 (0.1 g, 0.4 mmol) is heated under reflux in HCl-saturated methanol at 70° C. for 5 h. The solvent is removed under reduced pressure using a rotary evaporator.

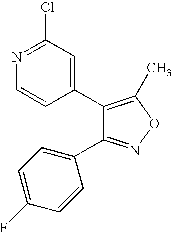

MS m/z (%) 288, 273, 246, 232, 220, 210, 184, 124, 95, 75

### 2-Ethoxy-4-[5-(4-fluorophenyl)isoxazol-4-yl]pyridine (23)

20 (0.1 g, 0.4 mmol) is heated under reflux in HCl-saturated ethanol at 70° C. for 5 h. The solvent is removed under reduced pressure using a rotary evaporator.

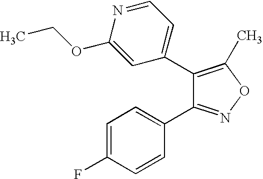

MS m/z (%) 298, 283, 254, 241, 228, 213, 199, 184, 106, 95, 75, 63, 51

1-9. (canceled)

10. A substituted isoxazole derivative of the formula I 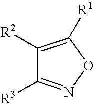
in which
R¹ is selected from the group consisting of
a) H;
b) C₁-C₆-alkyl which may have 1 or 2 substituents independently of one another selected from the group consisting of NR⁴R⁵ and OR⁶;
c) an aromatic or nonaromatic heterocycle having 5 or 6 ring atoms, including 1, 2 or 3 heteroatoms, independently of one another selected from the group consisting of N, O and S, where the heterocycle may have 1 or 2 substituents independently of one another selected from the group consisting of C₁-C₆-alkyl, halogen, CF₃, OR⁶, NR⁷R⁸, NR⁹COR¹⁰, a radical of the formula II 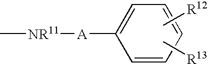
and a radical of the formula III 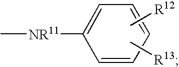
d) phenyl which may have 1, 2 or 3 substituents independently of one another selected from the group consisting of NR⁷R⁸, OR⁶, C₁-C₆-alkyl, halogen, CF₃, CN, NO₂ and CO₂R⁶;
e) phenyl-C₁-C₄-alkyl;
f) C₃-C₈-cycloalkyl; and
g) NR⁷R⁸;
one of the radicals R² and R³ is a radical of the formula IV 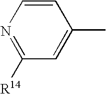
in which R¹⁴ is C₁-C₆-alkyl, halogen, CF₃, OR⁶, NR⁷R⁸, NR⁹COR¹⁰, a radical of the formula 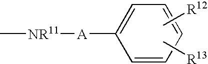
or a radical of the formula 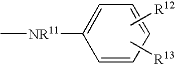
and
the second of the radicals R² and R³ is 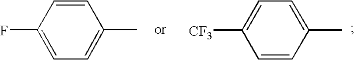
R⁴ and R⁵ independently of one another are H, C₁-C₆-alkyl, phenyl or phenyl-C₁-C₄-alkyl or together with the nitrogen atom to which they are attached form a saturated 5- or 6-membered heterocycle having 1 or 2 heteroatoms independently of one another selected from the group consisting of N and O;
R⁶, R⁷ and R⁸ independently of one another are H or C₁-C₆-alkyl;
R⁹ is H, C₁-C₆-alkyl or benzyl;
R¹⁰ is C₁-C₆-alkyl, C₃-C₆-cycloalkyl or phenyl which may have 1 or 2 substituents independently of one another, selected from the group consisting of C₁-C₆-alkyl, C₁-C₆-alkoxy and halogen;
R¹¹ is H, C₁-C₆-alkyl or phenyl-C₁-C₄-alkyl;
R¹² and R¹³ independently or one another are H, halogen, C₁-C₆-alkyl or C₁-C₆-alkoxy; and
A is straight-chain or branched C₁-C₆-alkylene; or
an optical isomer or a physiologically acceptable salt thereof.

11. The compound as claimed in claim 10 where R² is 4-fluorophenyl and R³ is the radical of the formula IV.

12. The compound as claimed in claim 10 where R² is the radical of the formula IV and R³ is 4-fluorophenyl.

13. The compound as claimed in claim 10 where R¹⁴ is selected from the group consisting of halogen, OH, NR⁷R⁸ and NR⁹COR¹⁰, where R⁷ to R¹⁰ have the meanings given in claim 1.

14. The compound as claimed in claim 10 where R¹ is H, phenyl which may have 1 or 2 halogen substituents, NR⁷R⁸ or C₁-C₆-alkyl, where R⁷ and R⁸ have the meanings given in claim 1.

15. The compound as claimed in claim 10 where R¹ is C₁-C₆-alkyl which is substituted by NR⁴R⁵ or OR⁶, an aromatic heterocyclic radical having 5 or 6 ring atoms including 1 or 2 heteroatoms independently of one another selected from the group consisting of N and O, where the heterocycle is optionally substituted by NR⁹COR¹⁰, phenyl which is optionally substituted by NR⁷R⁸ or C₁-C₆-alkoxy, NR⁷R⁸ or C₃-C₆-cycloalkyl.

16. A pharmaceutical composition comprising at least one compound as claimed in claim 10, if appropriate together with one or more pharmaceutically acceptable carriers and/or additives.

17. A method for treating immunologically mediated inflammatory diseases, wherein an amount of a compound of the formula I as claimed in claim 10 sufficient to have immunomodulating action and/or to inhibit the release of cytokine is administered to a person in need of such a treatment.
